(19) United States
(12) Patent Application Publication
(10) Pub. No.: US 2010/0120084 A1
(43) Pub. Date: May 13, 2010

(54) REACTION MEDIUM FOR IDENTIFYING/DETECTING MICROORGANISMS

(75) Inventors: El Mustapha Belgsir, Poitiers (FR); Yves Cenatiempo, Saint Julien I'Ars (FR); Manilduth Ramnath, Bruxerolles (FR); Denis Robichon, Blyes (FR)

Correspondence Address: OLIFF & BERRIDGE, PLC, P.O. BOX 320850, ALEXANDRIA, VA, 22320-4850, US

(73) Assignees: Biomerieux, Marcy L'Etoile (FR); Biocydex, Poitiers (FR)

(21) Appl. No.: 12/450,603

(22) PCT Filed: Apr. 28, 2008
(86) PCT No.: PCT/FR2008/050761

§ 371 Date: Oct. 2, 2009

(30) Foreign Application Priority Data
Apr. 30, 2007 (FR) 0754806

(51) Int. Cl. C12Q 1/04

(52) U.S. Cl. 435/34

(57) ABSTRACT

The present invention relates to a reaction medium for identifying/detecting microorganisms, that comprises at least one active molecule encapsulated in a cyclodextrin.

The field of the invention is that of the detection and identification of microorganisms, such as, in particular, bacteria or yeasts, by inoculation of reaction media.

A very large number of reaction media for detecting microorganisms currently exist. This detection may be based especially on the use of particular substrates, specific for an enzyme of the microorganism that it is desired to detect. Thus, in the case of bacteria, Escherichia coli strains are often demonstrated by revealing an enzyme activity of the osidase type, such as beta-glucuronidase or beta-galactosidase activity. Similarly, the Listeria genus can be detected by demonstrating a beta-glucosidase activity. An aminopeptidase activity may also be used to reveal a group, a genus or a species of bacteria. Thus, alanine-aminopeptidase activity, for example, makes it possible to differentiate Gram-negative bacteria from Gram-positive bacteria. Finally, mention may also be made of the detection of an esterase activity for, in particular, demonstrating the Salmonella genus. In addition to the use of particular substrates, the identification of a microorganism or of a group of microorganisms by virtue of reaction media may be based on the resistance of a microorganism to a therapeutic treatment. The medium then generally comprises one or more “active” molecules, such as, in particular, antibiotics against which the microorganism is liable to be resistant. Finally, media exist which are dedicated to the control of the environment, such as surface control, making it possible to detect the presence of microorganisms on a laboratory bench, for example. In order to detect the presence of microorganisms in a laboratory for the production of antibiotics, it is possible to use a medium comprising an active molecule such as beta-lactamase in order to inhibit the action of the residual antibiotics that might be present on the bench, and thus to allow the growth and identification of the possible microorganisms.

However, the maintaining of active molecules in solution is conditioned by their stability in complex media or at very high dilutions. They may be rapidly denatured depending on the physicochemical conditions, or degraded by enzymes. β-Lactamase, for example, is sensitive to heat denaturation (60˜70° C.). Antibiotics are also sensitive to heat. In order to compensate for this degradation over time, the initial concentration of active molecule must be very high, thereby posing a problem of production cost. The storage of media comprising such active molecules remains a major problem in this field of activity; they generally cannot be stored at ambient temperature, since sustained exposure to heat can induce denaturation of the active molecules of the medium, such as antibiotics, enzymes, etc. Furthermore, even if the cold chain is observed, the reaction media should generally be used within a period of 2 to 4 months following production thereof. It is therefore very important to increase the stability of the active molecules present in a reaction medium.

The present invention therefore proposes to improve the reaction media for the detection of microorganisms currently marketed by decreasing their production cost and improving their stability, so as to prolong their shelf life.

Surprisingly, the inventors have shown that the encapsulation of active molecules in cyclodextrins makes it possible to protect these active molecules in reaction media, conferring resistance of the active molecules to various factors such as heat, shaking, etc.

Cyclodextrins or cycloamyloses are well-known molecules. They are composed of a hydrophobic cavity in which hydrophobic molecules can be lodged, and of a hydrophilic outer face enabling the cyclodextrin-hydrophobic molecule complex to dissolve in aqueous solvents. By virtue of this apolar cavity, cyclodextrins are capable of forming inclusion complexes in an aqueous medium with a large variety of hydrophobic host molecules. The result of this complexation is the solubilization of hydrophobic molecules that are highly insoluble in the aqueous phase. However, these molecules have, to the applicant's knowledge, never been described as being capable of protecting active molecules in reaction media against various factors such as heat, shaking, etc. The use of active molecule(s)/cyclodextrin(s) encapsulation complexes in reaction media thus makes it possible to increase the stability of these active molecules and to prolong the lifetime of these reaction media.

Before proceeding with the description of the invention, the definitions below are given in order to facilitate the disclosure of the invention.

The term reaction medium is intended to mean a medium comprising all the elements necessary for the expression of a metabolism and/or for the growth of microorganisms. The reaction medium may be solid, semi-solid or liquid. The term “solid medium” is intended to mean, for example, a gelled medium. Agar is the conventional gelling agent in microbiology for the culture of microorganisms, but it is possible to use gelatin or agarose. A certain number of preparations are commercially available, for instance Columbia agar, Trypcase-soya agar, Mac Conkey agar, Sabouraud agar or, more generally, those described in the Handbook of Microbiological Media (CRC Press). The reaction medium may comprise one or more elements in combination, such as amino acids, peptones, carbohydrates, nucleotides, minerals, vitamins, active molecules such as antibiotics, enzymes, surfactants, buffers, phosphate salts, ammonium salts, sodium salts, metal salts, one or more substrates enabling the detection of an enzyme activity or metabolic activity, etc.

The medium may also comprise a colorant. By way of indication, as a colorant, mention may be made of Evans blue, neutral red, sheep blood, horse blood, an opacifier such as titanium oxide, nitroaniline, malachite green, brilliant green, etc.

The reaction medium may be a revealing medium, or a culture and revealing medium. In the first case, the microorganisms are cultured before inoculation and, in the second case, the detection and/or identification medium also constitutes the culture medium.

For the purpose of the present invention, the term microorganism covers bacteria, in particular Gram-negative bacteria and Gram-positive bacteria, yeasts, and, more generally, organisms, generally single-cell organisms, that are invisible to the naked eye, and that can be multiplied and manipulated in the laboratory.

By way of Gram-negative bacteria, mention may be made of bacteria of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Branhamella, Neisseria, Burkholderia, Citrobacter, Hafnia, Edwardsiella, Aeromonas, Moraxella, Pasteurella, Providencia, Actinobacillus, Alcaligenes, Bordetella, Cedecea, Erwinia, Pantoea, Ralstonia, Stenotrophomonas, Xanthomonas and Legionella.

By way of Gram-positive bacteria, mention may be made of bacteria of the following genera: Enterococcus, Streptococcus, Staphylococcus, Bacillus, Listeria, Clostridium, Gardnerella, Kocuria, Lactococcus, Leuconostoc, Micrococcus, Mycobacteria and Corynebacteria.

By way of yeasts, mention may be made of yeasts of the following genera: Candida, Cryptococcus, Saccharomyces and Trichosporon.

The term active molecule is intended to mean a molecule which produces an effect, such as a destructive capacity, on the microorganisms or a catalyst effect on chemical reactions, and which is degraded over time, under the effect of heat in particular.

The term “active molecule” is preferably intended to mean an antibiotic or an enzyme.

The term antibiotic is intended to mean a chemical substance which has a destructive capacity on microorganisms.

Mention may in particular be made of the family of beta-lactams, comprising in particular the penams (such as penicillin; bipenicillin; extencillin; oracillin, oxacillin; cloxacillin; ampicillin; amoxicillin; bacampicillin; metampicillin; pivampicillin; azlocillin; mezlocillin; piperacillin; ticarcillin; pivmecillinam; oxapenam; clavulanic acid; sulbactam; tazobactam); the penems (such as imipenem); the cephems (such as the 1st-generation cephalosporins (cefalexin; cefadroxil; cefaclor; cefatrizine; cefalotin; cefapyrine; cefazoline), the 2nd-generation cephalosporins (cefoxitin; cefamandole; cefotetan; cefuroxime), the 3rd-generation cephalosporins (cefotaxime; cefsulodine; cefoperazone; cefotiam; ceftazidime; ceftriaxone; cefixime; cefpodoxime; latamoxef)); the monobactams such as aztreonam.

Mention may also be made of the family of fosfomycins; glycopeptides (vancomycin; teicoplanin); polymycins (colistin); gramicidins and tyrocidin (bacitracin; tyrothricin); aminoglycosides (streptomycin; tobramycin; amikacin; sisomicin; dibekacin; netilmicin); macrolides (spiramycin; erythromycin; erythrocine; josamycin; roxithromycin; clarithromycin; azithromycin); lincosamides (lincomycin; clindamycin); synergistins (virginiamycin; pristinamycin); phenicols (chloramphenicol; thiamphenicol); tetracyclines (tetracycline; doxycycline; minocycline); fusidic acid; oxazolidinones (linezolide); rifamycins (rifamycin; rifampicin); quinolones (nalidixic acid; oxolinic acid; pipemidic acid); fluoroquinolones (flumequine; pefloxacin; norfloxacin; ofloxacin; ciprofloxacin; enoxacin; levofloxacin; moxifloxacin); oxyquinolines (nitroxoline; tilboquinol); nitrofurans (nitrofurantoin; nifuroxazide); nitroimidazoles (metronidazole; ornidazole); sulfamides (sulfadiazine; sulfamethisol), trimethoprims (trimethoprim).

The term enzyme is intended to mean a molecule that is protein in nature and that catalyzes the biochemical reactions of the metabolism occurring in the cellular or extracellular medium. Mention may in particular be made of oxidoreductases (such as oxidases, reductases, peroxidases, oxygenases, hydrogenases or dehydrogenases); transferases (such as kinases; transaminases; mutases); hydrolases (such as esterases; peptidases; osidases; glucosidases); lyases (such as decarboxylases, aldolases; dehydratases); isomerases (such as racemases; epimerases); ligases. Preferably, the enzyme is a hydrolase, and even more preferably a beta-lactamase.

The term cyclodextrin is intended to mean a molecule of the family of cyclic oligosaccharides composed of α-(1,4)-linked glucopyranose subunits and corresponding to the empirical formula C₄₂H₇₀O₃₅ or to a derivative of this molecule, in which the hydroxyl groups of the glucopyranose units may be aminated, esterified or etherified. Mention may in particular be made of beta-cyclodextrin (BCD), hydroxypropyl-beta-cyclodextrin (HPCD), methyl-beta-cyclodextrin (MCD), alpha-cyclodextrin (ACD) and gamma-cyclodextrin (GCD).

Preferably, the cyclodextrin is chosen from an alpha-cyclodextrin, which is preferably cyclomaltohexaose (bioCydex reference ACD N0; CAS No. 51211-54-9); a gamma-cyclodextrin, which is preferably cyclomaltooctaose (bioCydex reference GCD N0; CAS No. 91464-90-3); a beta-cyclodextrin, which is preferably a 2-O-methyl-beta-cyclodextrin or randomly 2-O-methyl-cyclomaltoheptaose (bioCydex reference BCD C15); or preferably a 2-hydroxypropyl-beta-cyclodextrin, also known as randomly 2,3,6-O-(2-hydroxypropyl)cyclomaltoheptaose (bioCydex reference BCD R59, CAS No. 128449-35-5); or preferably a monopropanediamino-beta-cyclodextrin, also known as 6^{I}-(3-aminopropylamino)-6^{I}-deoxycyclomaltoheptaose (bioCydex reference BCD A56). In general, the cyclodextrins of the present invention were provided by the company BioCydex (Poitiers, France). By way of indication, the cyclomaltohexaose and the cyclomaltooctaose are sold by Wacker Chemie; the cyclomaltoheptaose, the 2-O-methylcyclomaltoheptaose and the hydroxypropyl-beta-cyclodextrin are sold by Roquette Frères, and the monopropanediamino-beta-cyclodextrin is sold by BioCydex. The expression substrate enabling the detection of an enzyme activity or metabolic activity is intended to mean any molecule capable of directly or indirectly generating a detectable signal due to an enzyme activity or metabolic activity of the microorganism. When this activity is an enzyme activity, reference is then made to an enzyme substrate. The term “enzyme substrate” is intended to mean any substrate than can be hydrolyzed by an enzyme so as to give a product which enables the direct or indirect detection of a microorganism. This substrate comprises in particular a first portion specific for the enzyme activity to be revealed and a second portion which acts as a label, hereinafter referred to as label portion. This label portion may be chromogenic, fluorogenic, luminescent, etc. As chromogenic substrate well suited to solid supports (filter, agar, electrophoresis gel), mention may in particular be made of substrates based on indoxyl and its derivatives, and substrates based on hydroxyquinoline or esculetin and their derivatives, which enable the detection of osidase and esterase activities.

By way of indoxyl-based substrates, mention may in particular be made of 3-indoxyl, 5-bromo-3-indoxyl, 5-iodo-3-indoxyl, 4-chloro-3-indoxyl, 5-bromo-4-chloro-3-indoxyl, 5-bromo-6-chloro-3-indoxyl, 6-bromo-3-indoxyl, 6-chloro-3-indoxyl, 6-fluoro-3-indoxyl, 5-bromo-4-chloro-N-methyl-3-indoxyl, N-methyl-3-indoxyl, etc.

Mention may also be made of flavoid-derived substrates, such as, in particular, 3′,4′-dihydroxyflavone-4′-β-D-riboside, 3′,4′-dihydroxyflavone-4′-β-D-galactoside, 3′,4′-dihydroxyflavone-4′-β-D-glucoside, 3-hydroxyflavone-β-D-galactoside, 3-hydroxyflavone-β-D-glucoside or 3′,4′-dihydroxyflavone-3′,4′-diacetate.

Mention may also be made of substrates based on nitrophenol (ortho-nitrophenol, para-nitrophenol, etc.) and nitroaniline and derivatives, which make it possible to detect osidase and esterase activities in the case of nitrophenol-based substrates, and peptidase activities in the case of nitroaniline-based substrates.

Finally, mention may be made of substrates based on naphthol and naphthylamine and their derivatives, which make it possible to detect osidase and esterase activities by means of naphthol, and peptidase activities by means of naphthylamine. This substrate may in particular, but in a nonlimiting manner, enable the detection of an enzyme activity such as the activity of an osidase, peptidase, esterase, etc.

Mention may also be made of substrates based on coumarin and derivatives, which also make it possible to detect osidase and esterase activities in the case of substrates based on hydroxycoumarins, and in particular 4-methylumbelliferone or cyclohexenoesculetin, and peptidase activities in the case of substrates based on aminocoumarins, and in particular 7-amino-4-methylcoumarin.

Mention may also be made of substrates based on aminophenol and derivatives, which make it possible to detect osidase, esterase and peptidase activities. Mention may also be made of substrates based on alizarin and derivatives, which make it possible to detect osidase and esterase activities. Finally, mention may be made of substrates based on naphthol and naphthylamine and their derivatives, which make it possible to detect osidase and esterase activities by means of naphthol, and peptidase activities by means of naphthylamine.

The term “substrate based on naphthol” is intended to mean in particular substrates based on α-naphthol, on β-naphthol, on 6-bromo-2-naphthol, on naphthol AS BI, on naphthol AS, or on p-naphtholbenzein, as described in patent application EP1224196 by the applicant. This may be osidase, esterase, phosphatase or sulfatase substrates. The osidase substrates are in particular N-acetyl-β-hexosaminidase, β-galactosidase, α-galacotosidase, β-glucosidase, α-glucosidase, β-glucuronidase, β-cellobiosidase or α-mannosidase substrates.

The term “substrate based on alizarin” is intended to mean in particular the substrates described in patent EP1235928 by the applicant.

The enzymatic substrate may also be a natural substrate, the hydrolysis product of which is detected directly or indirectly. As a natural substrate, mention may in particular be made of tryptophan for detecting tryptophanase or deaminase activity, a cyclic amino acid (tryptophan, phenylalanine, histidine, tyrosine) for detecting deaminase activity, phosphatidyl inositol for detecting phospholipase activity, etc. When this activity is a metabolic activity, the substrate is then a metabolic substrate, such as a source of carbon or of nitrogen, coupled to an indicator that produces a coloration in the presence of one of the products of the metabolism.

By way of indication, the substrates used for detecting a beta-glucuronidase activity may in particular be 4-methylumbelliferyl-beta-glucuronide, 5-bromo-4-chloro-3-indolyl-beta-glucuronide, 5-bromo-6-chloro-3-indolyl-beta-glucuronide, 6-chloro-3-indolyl-beta-glucuronide, alizarin-beta-glucuronide, cyclohexenoesculetin-beta-glucuronide or their salts. By way of indication, the substrates used for detecting a beta-galactosidase activity may in particular be 4-methylumbelliferyl-beta-galactoside, 5-bromo-4-chloro-3-indolyl-beta-galactoside, 5-bromo-6-chloro-3-indolyl-beta-galactoside, 6-chloro-3-indolyl-beta-galactoside, alizarin-beta-galactoside, cyclo-hexenoesculetin-beta-galactoside or their salts. The substrates used for detecting a beta-glucosidase activity may in particular be 4-methylumbelliferyl-beta-glucoside, 5-bromo-4-chloro-3-indolyl-beta-glucoside, 5-bromo-6-chloro-3-indolyl-beta-glucoside, 6-chloro-3-indolyl-beta-glucoside, alizarin-beta-glucoside, cyclohexenoesculetin-beta-glucoside, nitrophenyl-beta-glucoside, dichloroaminophenylglucoside or their salts.

The term biological sample is intended to mean a clinical sample, derived from a specimen of biological fluid, or a food sample, derived from any type of food. This sample may thus be liquid or solid and mention may be made, in a nonlimiting manner, of a clinical blood, plasma, urine or feces sample, nose, throat, skin, wound or cerebrospinal fluid specimens, a food sample from water, from drinks such as milk or a fruit juice; from yogurt, from meat, from eggs, from vegetables, from mayonnaise, from cheese; from fish, etc., a food sample derived from a feed intended for animals, such as, in particular, a sample derived from animal meals.

In this respect, the invention relates to a reaction medium for identifying/detecting microorganisms, that comprises at least one active molecule encapsulated in a cyclodextrin.

According to one preferred embodiment of the invention, the cyclodextrin is chosen from:

an alpha-cyclodextrin, which is preferably cyclomaltohexaose (bioCydex reference ACD N0; CAS No. 51211-54-9); a gamma-cyclodextrin, which is preferably cyclomaltooctaose (bioCydex reference GCD N0; CAS No. 91464-90-3); a beta-cyclodextrin, which is preferably a 2-O-methyl-beta-cyclodextrin or randomly 2-O-methylcyclomaltoheptaose (bioCydex reference BCD C15); or preferably a 2-hydroxypropyl-beta-cyclodextrin, also known as randomly 2,3,6-O-(2-hydroxypropyl)cyclomaltoheptaose (bioCydex reference BCD R59; CAS No. 128449-35-5); or preferably a monopropanediamino-beta-cyclodextrin, also known as 6^{I}-(3-aminopropylamino)-6^{I}-deoxycyclomaltoheptaose (bioCydex reference BCD A56).

According to one preferred embodiment of the invention, the active molecule is an antibiotic. Preferably, the antibiotic is chosen from the penam or cepham family. Preferably, the antibiotic is cefoxitin or cloxacillin.

Of course, the reaction medium according to the present invention may comprise one antibiotic or several antibiotics. Those skilled in the art will easily adjust the concentration of antibiotic according to the desired effect. Preferably, the concentration of antibiotic is between 0.01 and 80 mg/l, preferably between 0.05 and 32 mg/l, even more preferably between 0.1 and 8 mg/l, and even more preferably between 0.25 and 6 mg/l.

By way of indication, when the antibiotic is cefotaxime the concentration of cefotaxime in the medium is preferably between 0.25 and 8 mg/l, preferably between 1 and 2 mg/l; when the antibiotic is cefoxitin, the concentration of cefoxitin in the medium is preferably between 0.1 and 8 mg/l, and even more preferably between 0.25 and 6 mg/l; when the antibiotic is cloxacillin, the concentration of cloxacillin in the medium is preferably between 0.1 and 8 mg/l, and even more preferably between 0.25 and 6 mg/l; when the antibiotic is ceftazidime, the concentration of ceftazidime in the medium is preferably between 0.25 and 8 mg/l, preferably between 2 and 2.5 mg/l; when the antibiotic is ceftriaxone, the concentration of ceftriaxone in the medium is preferably between 0.25 and 8 mg/l, preferably between 1 and 2.5 mg/l; when the antibiotic is cefpodoxime, the concentration of cefpodoxime in the medium is preferably between 0.1 and 32 mg/l, preferably between 0.75 and 10 mg/l, and even more preferably between 1 and 6 mg/l.

According to one preferred embodiment of the invention, the active molecule is an enzyme, preferably beta-lactamase. Those skilled in the art will adjust the concentration of enzyme according to the desired effect. Preferably, the concentration of beta-lactamase in the medium is preferably between 50 and 500 IU/l, preferably between 100 and 150 IU/l.

According to one preferred embodiment of the invention, the reaction medium comprises at least one substrate enabling the detection of an enzyme activity or metabolic activity.

The medium may also comprise a combination of substrates, according to the microorganisms that it is desired to identify. Those skilled in the art will adjust the concentration of substrate(s) according to the microorganism that it is desired to identify. Preferably, the concentration of substrate is between 25 and 750 mg/l, preferably between 40 and 200 mg/l. By way of indication, when the substrate 5-bromo-4-chloro-3-indolyl-β-D-glucoside enabling the detection of a beta-glucosidase enzyme activity is used, the concentration is preferably between 25 and 500 mg/l, preferably between 40 and 150 mg/l. By way of indication, when the substrate 5-bromo-4-chloro-3-indolyl-N-acetyl-β-D-glucosaminide enabling the detection of a hexosaminidase enzyme activity is used, the concentration is preferably between 25 and 500 mg/l, preferably between 40 and 150 mg/l. By way of indication, when 5-bromo-6-chloro-3-indolylphosphate enabling the detection of a phosphatase activity is used, the concentration is preferably between 25 and 750 mg/l, preferably between 40 and 200 mg/l.

Those skilled in the art may also use a biplate, making it possible to readily compare two media, comprising various substrates, onto which the same biological sample will be deposited.

Preferably, said substrate enabling the detection of an enzyme activity or metabolic activity is an enzyme substrate, preferably a fluorescent or chromogenic enzyme substrate.

Preferably, the enzyme activity is chosen from the following enzyme activities: osidase, esterase and peptidase, and more preferably said same enzyme activity is chosen from the following enzyme activities: βB-D-glucosidase, β-D-galactosidase, alpha-D-glucosidase, alpha-D-galactosidase, alpha-mannosidase, β-D-glucuronidase, N-acetyl-β-D-hexosaminidase, β-D-cellobiosidase, esterase, phosphatase, phospholipase, sulfatase and peptidase.

The invention also relates to a method for detecting and/or identifying microorganisms, characterized in that it comprises the following steps consisting in:
a) providing a reaction medium as defined above,
b) inoculating the medium with a test biological sample,
c) allowing to incubate, and
d) detecting and/or identifying the microorganisms.

The inoculating of the microorganisms can be carried out by any of the inoculation techniques known to those skilled in the art. An incubation step may be carried out at a temperature for which the enzyme activity that it is desired to detect is optimal, that can be readily chosen by those skilled in the art according to the enzyme activity to be detected. Step d) can be carried out by means of a visual examination, by colorimetry or by fluorimetry.

The invention also relates to the use of the reaction medium as defined above, for detecting and/or identifying microorganisms.

The invention also relates to the use of cyclodextrin(s) for increasing the stability of a reaction medium. By virtue of such a use, it is possible to put back the expiry date of the reaction medium, and to store the medium more readily.

Preferably, the cyclodextrin used is chosen from an alpha-cyclodextrin, which is preferably cyclomaltohexaose (bioCydex reference ACD N0; CAS No. 51211-54-9); a gamma-cyclodextrin, which is preferably cyclomaltooctaose (bioCydex reference GCD N0; CAS No. 91464-90-3); a beta-cyclodextrin, which is preferably a 2-O-methyl-beta-cyclodextrin or randomly 2-O-methylcyclomaltoheptaose (bioCydex reference BCD C15); or preferably a 2-hydroxypropyl-beta-cyclodextrin, also known as randomly 2,3,6-O-(2-hydroxypropyl)cyclomaltoheptaose (bioCydex reference BCD R59; CAS No. 128449-35-5); or preferably a monopropanediamino-beta-cyclodextrin, also known as 6^{I}-(3-aminopropylamino)-6^{I}-deoxycyclomaltoheptaose (bioCydex reference BCD A56).

The invention also relates to the use of cyclodextrin(s) for protecting active molecules against physicochemical degradation in a reaction medium, such as, in particular, heat or shaking. Preferably, the cyclodextrin used is chosen from an alpha-cyclodextrin, which is preferably cyclomaltohexaose (bioCydex reference ACD N0; CAS No. 51211-54-9); a gamma-cyclodextrin, which is preferably cyclomaltooctaose (bioCydex reference GCD N0; CAS No. 91464-90-3); a beta-cyclodextrin, which is preferably a 2-O-methyl-beta-cyclodextrin or randomly 2-O-methylcyclomaltoheptaose (bioCydex reference BCD C15); or preferably a 2-hydroxypropyl-beta-cyclodextrin, also known as randomly 2,3,6-O-(2-hydroxypropyl)cyclomaltoheptaose (bioCydex reference BCD R59; CAS No. 128449-35-5); or preferably a monopropanediamino-beta-cyclodextrin, also known as 6^{I}-(3-aminopropylamino)-6^{I}-deoxycyclomaltoheptaose (bioCydex reference BCD A56).

The examples below are given by way of explanation and are in no way limiting in nature. They will make it possible to understand the invention more clearly.

The examples below relate to the protection against degradation of 3 active molecules (two antibiotics, cloxacillin and cefoxitin, and one enzyme, β-lactamase) by virtue of their encapsulation in a cyclodextrin. Several cyclodextrins (ACD NO, GCD N0, BCD C15, BCD R59, BCD A56) of the Solvamax® range were tested for their ability to slow down the heat degradation of active molecules. The stability was evaluated by HPLC.

### 1. Protection and Stabilization of Cloxacillin

Cloxacillin (molecular mass 475.88) is an antibiotic of β-lactam type, used to inhibit the growth of certain bacterial species such as Enterobacter aerogenes or Escherichia coli. The cyclodextrins of the BioCydex library were incubated with cloxacillin in a 1:1 molar ratio (concentration=210.12 mM), at 20° C. for 72 h (shaking in the dark). The amount of residual cloxacillin was then analyzed by HPLC.

The following tests were carried out in an aqueous medium and at high temperature (75° C.) in order to accelerate the rate of degradation.

1.1 Screening ex-situ

The sodium salt of cloxacillin (Sigma, Ref. C 9393) has a high intrinsic solubility in aqueous medium (˜100 g·L⁻¹).

The complexes were prepared at ambient temperature with cloxacillin:cyclodextrin molar ratios of 1:16 and of 1:79. The solutions containing 0.63 mM, i.e. 300 mg·L⁻¹, of cloxacillin were incubated at 75° C. for 31 h. The analysis of the samples and the quantification thereof so as to evaluate the stability of the antibiotic were carried out on a Thermo Finnigan SpectraSYSTEM HPLC system equipped with a P1000XR pump, an AS3000 automatic injector, a UV1000 UV/visible detector, and a Merck Chromolith® Performance RP-18 endcapped column (100-4.6 mm) preceded by a Merck Chromolith® RP-18e guard column (5-4.6 mm).

The mobile phases were prepared using HPLC-grade acetonitrile and water acidified with trifluoroacetic acid (100 μL/L). A methanol/water gradient of 0/100 to 100/0 was applied in 12 min. After stabilization for 2 min, the column is reequilibrated under the initial conditions. The elution speed is 1 mL/min, the temperature is 22° C. and the detection is carried out at 220 nm. The sample injection volume is 20 μL. The chromatographic data were processed using the Atlas software, version 2003.1 (Thermo Electron Corporation, UK).

The results are given in table I:

TABLE I
Stability of cloxacillin in the presence of cyclodextrins at 75° C.

| Cyclodextrin | Cloxacillin stability (%) |
|---|---|
| None | 0.6 |
| BCD R59 10 mM | 16.3 |
| BCD R59 50 mM | 37.7 |
| BCD C15 10 mM | 14.9 |
| BCD C15 50 mM | 39.9 |

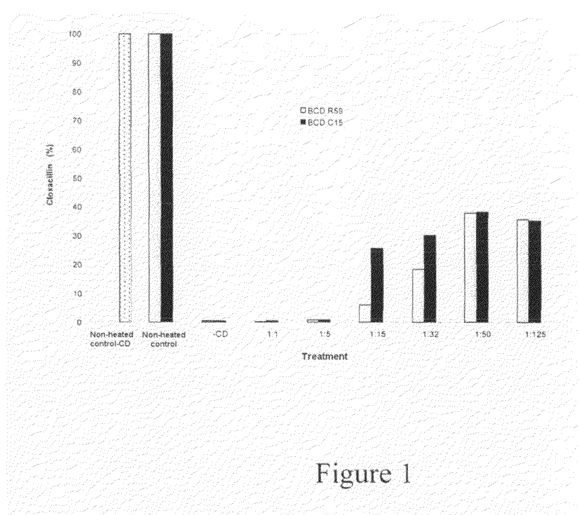

These results demonstrate the protective effect of the cyclodextrins BCD R59 and BCD C15 on cloxacillin. These results were confirmed under the same experimental conditions when the cloxacillin:cyclodextrin molar ratio was a minimum of 1:15. In this respect, FIG. 1 shows the protective effect of the cyclodextrins against heat-degradation of cloxacillin (−CD: absence of cyclodextrin; 1:1 to 1:125: cloxacillin:CD molar ratios).

The cyclodextrin BCD C15 provided the best protection at a low ratio. For molar ratios higher than 1:50, similar performance levels were observed for the two cyclodextrins. These results show that the complexation of cloxacillin with a cyclodextrin such as BCD R59 or BCD C15 confers, on the antibiotic molecule, protection against inactivation by heat treatment (31 h at 75° C.) since 40% of the native molecule is found after heating, against 0% when the cloxacillin is not associated with the cyclodextrin.

### 2. Cefoxitin Complexation

Cefoxitin is an antibiotic of the β-lactam family, which inhibits mucopeptide synthesis by the bacterial wall.

2.1 Screening ex-situ

0.444 mM of cefoxitin (Sigma, Ref. C4786-5G) and 8.88 mM of cyclodextrin (ACD N0 or GCD NO or BCD R59 or BCD C15), i.e. in a molar ratio of 1:20, were dissolved with stirring for one hour at ambient temperature. The mixture was then brought to 65° C. for 90 min. The integrity of the cefoxitin was then analyzed by HPLC. The analysis of the samples and the quantification thereof were carried out on a Thermo Finnigan SpectraSYSTEM HPLC system equipped with a P1000XR pump, an AS3000 automatic injector, a UV1000 UV/visible detector, and a Merck Chromolith® Performance RP-18 endcapped column (100-4.6 mm) preceded by a Merck Chromolith® RP-18e guard column (5-4.6 mm).

The mobile phases were prepared using HPLC-grade acetonitrile and water acidified with trifluoroacetic acid (100 μL/L). A methanol/water gradient of 0/100 to 100/0 was applied in 12 min. After stabilization for 2 min, the column is reequilibrated under the initial conditions. The elution speed is 1 mL/min, the temperature is 22° C. and the detection is carried out at 254 nm. The sample injection volume is 20 μL. The chromatographic data were processed using the Atlas software, version 2003.1 (Thermo Electron Corporation, UK).

The results are given in table II:

TABLE II
Effect of various cyclodextrins on the
stability of cefoxitin at 65° C.

| Cyclodextrin | Cefoxitin stability/% |
|---|---|
| None | 38.5 |
| ACD N0 | 41.3 |
| GCD N0 | 40.5 |
| BCD R59 | 43.8 |
| BCD C15 | 42.0 |

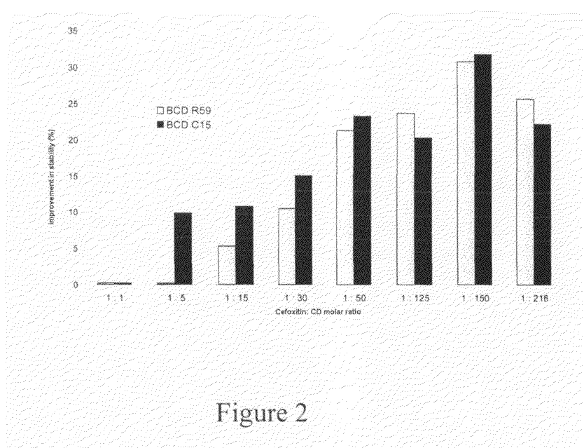

The optimization of protection was sought by varying the cefoxitin:cyclodextrin (BCD R59 and BCD C15) molar ratio from 1:1 to 1:216, the other conditions remaining unchanged. In this respect, FIG. 2 shows the protective effect of BCD R59 and of BCD C15 on cefoxitin in an aqueous medium. The improvement in stability was calculated relative to the control without cyclodextrin.

For molar ratios ranging between 1:6 and 1:30, the cyclodextrin BCD C15 was the most effective. Above this, the two cyclodextrins were equivalent. A molar ratio of 1:50 was sufficient to achieve a substantial degree of protection at low temperature. In this case, the stability of cefoxitin after 90 min at 65° C. is 47.5% instead of 38.5% with the control without cyclodextrin.

These results demonstrate that the cyclodextrins ACD NO, GCD N0, BCD R59 and BCD C15 protect cefoxitin against heat denaturation since, in the presence of BCD C15, the complexed antibiotic is approximately 10% less degraded than the molecule alone.

### 3. Protection of Beta-Lactamase

3.1 Stability of β-lactamase

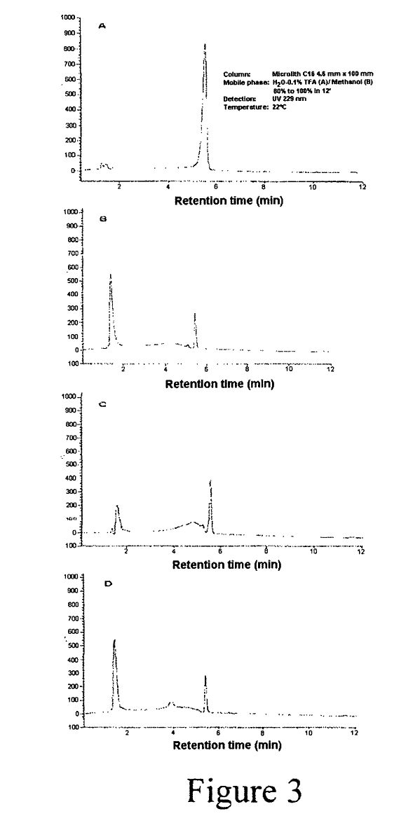

β-Lactamase (Genzyme Biochemicals Ref. BELA-70-1431) in an amount equivalent to 0.375 U·mL⁻¹ was mixed with the cyclodextrin BCD A56 of the Protéosol® range (1 or 10 mM), at ambient temperature and in the dark, for 30 min. The mixture was then heated at 70° C. for 45 min. Amoxicillin (Glaxo, Ref. 5003), 500 mg·L⁻¹, which reveals β-lactamase activity, was then added at ambient temperature. After incubation for 5 min at 25° C., the residual amoxicillin was analyzed by HPLC. The profiles obtained are given in FIG. 3, which shows the HPLC analysis of the amoxicillin (A: untreated amoxicillin control; B: amoxicillin+β-lactamase not heated; C: amoxicillin+β-lactamase heated; D: amoxicillin+β-lactamase: BCD A56 complex (1 mM) heated. T_{R} amoxicillin=5.5 min). The amoxicillin peak came off at 5.5 min.

The activity of the β-lactamase was attested to by the decrease in the amoxicillin peak (FIG. 3B) compared with the amoxicillin alone control (FIG. 3A) and by the appearance of hydrolysis products characterized by an unresolved peak at shorter retention times. When the β-lactamase was degraded by heating, it lost some of its activity, which was reflected by better stability of the amoxicillin (FIG. 3C). The same heat treatment in the presence of cyclodextrin (FIG. 3D) made it possible to maintain a β-lactamase activity identical to that of the amoxicillin-β-lactamase non-heated control (FIG. 3B).

On the basis of the above profiles, it was possible to quantify the concentration of amoxicillin and to link this value to the β-lactamase activity. The results are given in table III.

TABLE III
Influence of BCD A56 on β-lactamase
resistance to heat degradation

| | [Amoxicillin] |
|---|---|
| Treatment of β-lactamase | (arbitrary units) |
| Heating without BCD A56 | 100 |
| Heating + BCD A56 at 1 mM | 4.3 |
| Heating + BCD A56 at 10 mM | 0.8 |

Taking the situation in the absence of cyclodextrin as reference, it was concluded that there was excellent protection of the β-lactamase in the presence of BCD A56 at 10 mM and that, in addition, this cyclodextrin did not interact with the active site of the enzyme.

These results demonstrate that the beta-lactamase associated with the cyclodextrin conserves all its enzyme activity even after heating at 70° C. for 45 minutes, since it remains capable of completely degrading amoxicillin, which is the substrate for the enzyme, unlike the uncomplexed protein. The cyclodextrin therefore protects the enzyme and its active site against denaturation owing to heat treatment.

### 4. Reaction Medium:

Two reaction media were produced from a ChromID™ MRSA medium, one with cefoxitin and the other containing a cyclodextrin BCD C15+cefoxitin complex. The latter was obtained by solubilizing the cefoxitin and the cyclodextrin in osmosed water and then stirring the solution at ambient temperature in the dark. The mixture was then filtered before being incorporated into agar.

The media are stored at 2-8° C. for 19 weeks and the performance levels (growth of MRSA: methicillin-resistant Staphylococcus aureus, inhibition of MSSA: methicillin-sensitive Staphylococcus aureus) are evaluated every week, compared with a commercially available ready-to-use medium.

The protection of cefoxitin by the cyclodextrin is measured by comparing the inhibition of MSSA growth (active cefoxitin) over time on the media with and without cyclodextrin BCD C15.

The MRSA and MSSA strains were inoculated on the media according to the three-quadrant streaking method and then the dishes were incubated for 48 h at 37° C.

The growth of the strains (presence of colonies on the dish) and also the color of the colonies after 24 and 48 h of incubation are observed. The results are given in the table below:

| | | | chromID MRSA + |
|---|---|---|---|
| | | chromID MRSA + | cefoxitin + |
| | | cefoxitin | cyclodextrin |
| Week | MRSA growth | + _{(2/2)} | + _{(2/2)} |
| 0 | MSSA growth | − _{(0/2)} | − _{(0/2)} |
| | colony color | G | G |
| Week | MRSA growth | + _{(2/2)} | + _{(2/2)} |
| 1 | MSSA growth | − _{(0/2)} | − _{(0/2)} |
| | colony color | G | G |
| Week | MRSA growth | + _{(2/2)} | + _{(2/2)} |
| 10 | MSSA growth | − _{(0/2)} | − _{(0/2)} |
| | colony color | G | G |
| Week | MRSA growth | + _{(2/2)} | + _{(2/2)} |
| 17 | MSSA growth | − _{(0/2)} | − _{(0/2)} |
| | colony color | G | G |
| Week | MRSA growth | + _{(1/1)} | + _{(1/1)} |
| 18 | MSSA growth | + _{(1/4 at 48 h 1 colony)} | − _{(0/4)} |
| | colony color | G | G |
| Week | MRSA growth | + _{(1/1)} | + _{(1/1)} |
| 19 | MSSA growth | + _{(2/4)} | + _{(2/4)} |
| | colony color | G | G |

Legend:
+: growth
−: absence of growth
(N): number of strains which grow/number of strains tested
G: green

As expected at T=0, the MRSA strains develop by forming green colonies, whereas the MSSA strains are correctly inhibited. The presence of cyclodextrins is compatible with use in a culture medium.

1. A reaction medium for identifying or detecting microorganisms, that comprises at least one active molecule encapsulated in a cyclodextrin.

2. The reaction medium as claimed in claim 1, wherein the cyclodextrin is chosen from: an alpha-cyclodextrin; a gamma-cyclodextrin, a beta-cyclodextrin; a 2-O-methyl-beta-cyclodextrin; a 2-hydroxypropyl-beta-cyclodextrin; and a monopropanediamino-beta-cyclodextrin.

3. The reaction medium as claimed in claim 1, wherein the active molecule is an antibiotic.

4. The reaction medium as claimed in claim 3, wherein the antibiotic is chosen from the penam or cepham family.

5. The reaction medium as claimed in claim 1, wherein the active molecule is an enzyme.

6. The reaction medium as claimed in claim 1, comprising at least one substrate enabling the detection of an enzyme activity or metabolic activity.

7. The reaction medium as claimed in claim 6, wherein said substrate enabling the detection of an enzyme activity or metabolic activity is an enzyme substrate.

8. A method for detecting and/or identifying microorganisms, comprising:
a) providing a reaction medium as claimed in claim 1,
b) inoculating the medium with a test biological sample,
c) allowing to incubate, and
d) detecting and/or identifying the microorganisms.

9. (canceled)

10. A method for increasing the stability of a reaction medium, comprising encapsulating at least one active molecule in a cyclodextrin.

11. A method for protecting active molecules against physiochemical degradation in a reaction medium, comprising encapsulating at least one active molecule in a cyclodextrin.
